(19) United States
(12) Patent Application Publication
(10) Pub. No.: US 2007/0059239 A1
(43) Pub. Date: Mar. 15, 2007

(54) Process for the preparation of radioactive labeled estradiol

(75) Inventors: Richard J. Baranczuk, (US); Anita Baranczuk, (US); Jay A. Spicer, Afton, OK (US)

Correspondence Address: LAW OFFICE OF JOHN C. MCMAHON, P.O. BOX 30069, KANSAS CITY, MO, 64112, US

(73) Assignee: Estradiol Images, LLC a Missouri limited liability company, Shawnee, KS (US)

(21) Appl. No.: 11/227,905

(22) Filed: Sep. 15, 2005

(51) Int. Cl. A61K 51/00; C07J 1/00

(52) U.S. Cl. 424/1.11; 552/625

(57) ABSTRACT

A method for producing a high yield of an estradiol labeled at the 16-alpha position thereof with a radioactive iodine, especially ¹²³I, for use as an imaging agent. The method includes the step of mixing a source of ¹²³I sodium iodine with 16-beta-bromoestradiol-17-beta and benzo-15-crown-5-ether in a water and organic solvent solution and heating under controlled temperature in the range from 90 to 120° Centigrade for sufficient time to react the iodide with the estradiol. Thereafter, resulting 16-alpha-123I iodoestradiol-17-beta is separated from the resultant mixture using high pressure liquid chromatography and then sterilized.

### BACKGROUND OF THE INVENTION

The present invention relates to radioactive imaging estradiol compounds and methods of producing compounds having high quality and with a high yield from precursor compounds and, in particular, is especially directed to the production of 16-alpha-123iodo-estradiol-17-beta.

### SUMMARY OF THE INVENTION

The inventors of the present invention were inventors or co-inventors of three U.S. Pat. Nos. 4,659,517; 4,855,125 and 4,882,141 which are directed to production and use of radioactive labeled estradiols. These patents are incorporated herein by reference.

While the processes described in the noted three patents were effective in producing a useable product, certain problems presented themselves, especially with respect to yield, and the present invention was developed to overcome these problems.

In particular, it is first noted that there are inherent problems in working with radioactive isotopes. A primary problem is that it is desirable for radioactive isotopes to have a short half life in the patient. In this manner, the patient can be dosed with sufficient radioactivity to provide an image shortly after injection, yet has only a small residual amount of radioactivity that decays in accordance with the half life of the material over a relative short time.

Thus, radioactive iodine 123 (I-123 or ¹²³I) is often considered desirable as an imaging agent as it has a half life of only about 13.3 hours. Thus, approximately two days after injection only about one sixteenth of the original amount of radioactivity remains in the patient.

While short half life imaging agents are highly preferable for patients, they present substantial problems with respect to manufacture and shipment. For example, I-123 can only be manufactured at certain select cyclotrons in North America and throughout the world. While several in North America could be used to make I-123, the product is currently being commercially made only in Vancouver, Canada. Even when other sources are available to make it, the number is low. For use, the manufactured I-123 must be packaged and shipped, as a radioactive hazard, to whatever location that will inject the patient. Somewhere, normally at the patient site, the I-123 must also be tagged or joined to the estradiol. In processes that were in the art prior to the earlier patents noted above, the processing time was so long (approximately 40 hours) that after shipment and subsequent manufacture, there was very little radioactive component left to the point of being useless for imaging. Furthermore, the I-123 is quite expensive, so much valuable material was lost just during the reaction time with the estradiol.

The above noted earlier patents of the present inventors significantly reduced the production time and made practical the use of the I-123 as an imaging agent. Nevertheless, certain problems still made the product less than fully satisfactory.

The major problem was that the yield of the process was comparatively low. For example, the yield in a typical reaction in accordance with the earlier patents was often on the order of 20 to 30 percent or even less. Because the radioactive component was quite expensive and time was important, it was neither desirable to dispose of 70 percent of the radioactive component nor to take the time to separate the components and rerun the reaction because the earlier made portion was then decaying. Consequently, it was desirable to have a method of production having a substantially greater yield in converting the estradiol derivative and radioactive component into a finished imaging agent.

A second lessor problem arises out of the spontaneous equilibrium reaction that modifies some iodide of the I-123 to iodate during manufacture, shipping, storage and the like. The iodate is not useful in the principal reaction with the estradiol. Therefore, it is also preferred to have a process that converts iodate to iodide and stabilizes the iodide to prevent it from further oxidation.

As perhaps only one patient at a facility is to be imaged, any excess radioactive component not needed for the patient must be disposed of as a radioactive hazard. Therefore, it is desirable to have a high yield and little left over radioactive component either as a precursor or final product.

### SUMMARY OF THE INVENTION

While prior art methods of production of 16-Alpha-123 iodoestradiol-17 Beta for use as an imaging agent have succeeded in reducing the production time so that the agent could be produced within about one half life of the radioactive iodine component or less, the yield was comparatively low and in the 30 percent or less range, such that either a large amount of the expensive and potentially hazardous ¹²³I was wasted or the process had to be repeated multiple times which took time and lost ¹²³I due to continued radioactive decay of the completed product. The present invention produces a high quality imaging agent wherein a yield of about 90 percent or better can be achieved in a relatively short time.

In particular, the present invention is highly effective in tagging or labeling an estradiol or estradiol derivative, preferably 16-beta-bromoestradiol-17-beta, with a halogen at the 16-alpha position, preferably iodine and most preferably ¹²³I and further preferably approximately 10 to 2000 millicuries of the iodine. While the invention is seen to be useful in forming such compounds as 16-alpha-haloestradiol-17-beta, 16-alpha-iodoestradiol-17-beta, 16-alpha-125iodoestradiol-17 beta and the like, the comparatively short production time and relatively high yield are seen to be especially favorable in the production of 16-alpha-123iodoestradiol-17-beta.

When used with radioactive ¹²³I, which is conventionally shipped as 123I-sodium iodide, the sodium iodide is first treated with an antioxidant such as an aqueous solution of sodium thiosulphate or sodium metabisulfite with heating to convert any oxidized sodium iodate which is not useful in the method to sodium iodide.

The resultant solution is dried and the 123I-sodium iodide separated from other remaining components, preferably by passage through a high pressure liquid chromatograph.

The pH of the collected 123I-sodium iodide is adjusted to be in the range preferably from about 7.1 to 8 and the moisture therein is removed. Thereafter, the resulting material is mixed with 16-beta-bromoestradiol-17-beta and a phase transfer agent, preferably benzo-15-crown-5-ether and dissolved in a solvent, preferably methyl ethyl ketone to form a mixture.

The resulting mixture is heated under controlled temperature in the range from 90 to 120° Centigrade, preferably in the range between 95 and 110° Centigrade and most preferably in the range from 100 to 105° Centigrade for approximately 0.5 to 1 hours, preferably 45 minutes. If the heating temperature drops below 90° Centigrade, the yield drops below 90 percent and, if the temperature is above 120° Centigrade, the reaction mixture degrades and is not usable.

Subsequent to heating, the mixture is subjected to a procedure to separate components, preferably by high pressure liquid chromatograph, so as to separate the radioactive iodine tagged estradiol from the remainder and then sterilized. Thereafter, the sterilized composition, preferably containing 16-alpha-123iodoestradiol-17-beta is mixed with a suitable injection medium and injected into a patient to allow radiation imaging of cancerous tumors that have an affinity for the estradiol compound.

### OBJECTS OF THE INVENTION

Therefore, in accordance with the invention, it is an object to produce an imaging product that uses a minimum of radioactive component during the manufacturing process; to provide a process to combine the radioactive component with an estradiol derivative as quickly as possible with a high yield of reaction; to provide such a process that produces a minimum of residual radioactive component; and to provide a process for production of a radioactive imaging agent that is comparatively easy to accomplish at a local lab, comparatively inexpensive and particularly suited for the intended usage thereof.

Other objects and advantages of this invention will become apparent from the following description wherein are set forth, by way of description and example, certain embodiments of this invention.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific details disclosed herein are not to be interpreted as limiting, but merely as a basis for the claims and as a representative basis for teaching one skilled in the art to variously employ the present invention in virtually any appropriately detailed method or process.

The method of the present invention is for the purpose of providing a comparatively very high yield of labeled estradiol with radioactive iodine at the 16 position on the estradiol (by high yield is meant that about 90 percent or greater of the starting ¹²³I reacts with the estradiol to produce the desired imaging compound).

The iodine may be any of the radioactive isotopes of iodine, but is preferably ¹²³I, as it has a comparatively short half life of approximately 13 hours and is suitable for use in imaging of cancerous tumors that have an affinity for the estradiol. Specifically, the tagged or labeled estradiol is injected into a patient and selectively binds to receptor sites on the tumor tissue. Thereafter, as the radioactive isotope decays, radiation is released by the decay that can be detected by a detector that in turn identifies the location from which the radiation initiated, so as to identify the precise situs and size of the tumor tissue in the patient. Not only does this locate the primary tumor, but it can also be used to locate any metastasis and/or to locate any small cancerous regions that are inadvertently missed during surgery to remove the primary tumor.

While the present invention is preferably utilized in making an ¹²³I labeled estradiol, it is foreseen that it may be used with other radioactive iodines, especially ¹²⁵I, or non radioactive iodines. Still further, it may be used to produce other haloestradiols, in particular from halides that with react with 16-beta-bromoestradiol-17-beta so as to join the halide to the estradiol at the 16 position.

¹²³I is not readily available and must be manufactured using a cyclotron configured for that purpose. While at any time there are numerous cyclotrons that may be configured to make ¹²³I with some modification, the only known cyclotron currently so configured in North America is in Vancouver, Canada and is operated by Nordion International which is then the most likely source of the ¹²³I in North America. Cyclotrons are found in other parts of the world, especially Europe, that can be used to manufacture ¹²³I.

While radioactive iodines are most often available as either sodium iodide or ammonia iodide, the sodium iodide is preferred in the present method, although it is foreseen that other iodide salts may be used in some circumstances.

The ¹²³I sodium iodide can be shipped in any convenient form such as liquid or preferably solid. Unfortunately, the sodium iodide oxidizes during manufacturing, shipping, storage, etc. to produce sodium iodate. The iodate form is not useful in accordance with the method of the invention, so it is preferred to convert all iodate to iodide prior to proceeding with the reaction with the estradiol in order to increase the yield of the radioactive iodine. Consequently, the sodium iodide is mixed with an antioxidant, preferably while heating, also preferably in the range from 105 to 110° Centigrade. The preferred antioxidant is sodium thiosulphate, although other antioxidants such as sodium metabisulfite may be used.

Subsequent to the process of converting at least a portion of the iodate to iodide, the resulting composition is preferably at least partially dried and then substantially purified. ¹²³I sodium iodide is preferably removed from other components of the composition by separating it from the other components in a high pressure liquid chromatograph. Other methods of separation are foreseeable, but the chromatograph is seen to be fast, efficient and highly effective at producing a generally purified ¹²³I sodium iodide. It is, of course, recognized that because this material is radioactive, that the composition starts changing immediately upon separation, but it provides a good starting point.

It was noted above that the iodide may be dried. Drying can be accomplished here and where called for below by such expedients as using a nitrogen gas blow through a container of the material to be dried, by heating (for example, at 100° Centigrade), by heating in a vacuum, or other suitable methods which will not degrade the components.

Subsequent to separation, the pH of the sodium iodide is adjusted to be in the range from 7.1 to 8.5 and preferably in the range from 7.5 to 8.0. In a preferred method, the pH is adjusted by first raising the pH to about 12 using sodium hydroxide and then reducing the pH to the preferred range of 7.5 to 8.0 using an acid such as ascorbic acid. The ascorbic acid acts also as an antioxidant to prevent oxidation to iodate and it is foreseen that other similar substances such as citric acid may be used for this purpose.

Estradiol is a hormone that is present in humans and other animals. As used herein the term estradiol refers to both estradiol and the many common derivatives or minor chemical modifications of estradiol that function in a manner similar, to estradiol and have affinity for the same receptor cites in animals, especially humans, as estradiol. In accordance with the invention, the estradiol is preferably brominated at the 16-beta position and is commonly known as 16-beta-bromoestradiol-17-beta.

The 16-beta-bromoestradiol-17-beta and ¹²³I sodium iodide are mixed with water and an organic a solvent and heated together. Excess estradiol is used to promote complete reaction of the iodide. The preferred organic solvent is methyl ethyl ketone although other solvents may be used. The water and solvent form a two phase aqueous (mainly with the iodide salt) and organic (mainly with the estradiol). The reaction temperature has been found to be important to the yield of the process and a temperature range of 90 to 120° Centigrade has been found to be highly effective. Below 90° Centigrade the yield drops below 90 percent and above 120° Centigrade, at least a portion of the components degrade. The reaction time varies with the particular reactants, temperature, etc., but it has been found that at least 90 percent yield can be obtained with approximately 45 minutes of reaction time; however, the time can vary and the preferred range is one half hour to one hour with the most preferred time being 45 minutes.

A phase transfer agent is also included in the reaction mixture and is preferably a crown ether, although any transfer agent suitable for working with the components of the mixture is seen to be suitable. The transfer agent helps in transferring the iodide to the organic phase for reaction with the estradiol. Most preferably, the transfer agent is benzo-15-crown-5-ether. It is believed that this particular crown ether also aids in the chemical reaction by separating the sodium and iodide ions, so that the iodide ion can more easily react with the estradiol. Certain other crown ethers that have been used in prior art processes have been found to provide lower yield, and it is believed that this occurs because they are less effective in separating the ions.

Subsequent to heating the reaction mixture, it is substantially dried and the resulting composition is injected into the high pressure liquid chromatograph. The chromatograph is preferably pretested with a non radioactive composition to determine when components peaks will occur. The resulting 16-alpha-123I-iodoestradiol-17-beta exits the chromatograph after the appropriate residence time of approximately twenty minutes.

The resultant relatively purified 16-alpha-123I-iodoestradiol-17-beta is then suitably prepared for injection into a patient.

The following example is provided for purposes of illustrating the invention and is not intended as a restriction upon the scope of the claims.

### EXAMPLE

In accordance with the invention, a 1000 millicurie sample of combined 123 iodo-sodium iodide (¹²³I—NaI) and 123 iodo-sodium iodate (¹²³I—NaIO₃) was placed in a vial with 250 micrograms of sodium thiosulphate dissolved in 100 microliters of water for the purpose of converting a sodium iodate present to sodium iodide. The mixture was heated in a stirred glycerine bath at a temperature of 105° centigrade for fifteen minutes.

Thereafter, the contents of the vial were removed from the bath and the interior of the vial was exposed to a nitrogen gas blow for the purpose of evaporating water from the contents of vial so as to leave a resulting mixture of 200 microliters in the vial for the purpose of reducing the mixture volume prior to injecting into a liquid chromatograph.

The resulting mixture was then injected in a high pressure liquid chromatograph at 600 pounds per square inch pressure wherein the chromatograph was of a type capable of dual solvent pumping and gradient equipped with both a UV detector and a radiation detector. The chromatograph had a reverse phase C18 silica gel 10 micron type column. The chromatograph was preset to pump at one milliliter per minute using a mixture of half water and half acetonitrile by weight as a carrier.

The effluent associated with the second peak of discharge from the chromatograph was collected in a vial. This peak had a residence time in the range from 2.9 to 3.5 minutes.

The pH of the contents of the vial was adjusted by the addition of 0.1 normal sodium hydroxide to 12 which required the addition of approximately 100 microliters. Thereafter, the pH was lowered by the addition of 0.1 molar ascorbic acid until a pH of 7.5 was obtained.

Thereafter, the contents of the vial were again placed on the heating bath and a blow of nitrogen gas was applied to evaporate essentially all moisture.

Subsequently, 800 micrograms of 16-beta-bromo-estradiol-17-beta and 500 micrograms of benzo-15-crown-5-ether were added to the vial and dissolved in 200 microliters of methyl ethyl ketone. Thereafter, 10 microliters of water was added to the vial and mixed after which the vial was placed again on the bath at a temperature of 105° centigrade for forty-five minutes.

The resulting composition was injected into the chromatograph upon which the ultraviolet detector was set for 280 microns and the radiation detector was activated. After approximately 20 minutes, the detectors were triggered and the effluent associated with a source peak was collected.

Thereafter, the component collected was filtered through a 0.2 micron non aqueous millipore filter and placed in a sterile vial.

A nitrogen gas blow through a sterile filter was used to evaporate all moisture to produce a dry product.

The dry product was dissolved in 1000 microliters of 90 percent by weight normal saline and 10 percent by weight absolute ethanol. A portion of the resulting mixture was thereafter injection into a patient for the purpose of imaging cancerous tumors that have an affinity (that is receptors) for estradiol.

It is to be understood that while certain forms of the present invention have been illustrated and described herein, it is not to be limited to the specific forms or arrangement of parts described and shown.

What is claimed and desired to be secured by Letters Patent is as follows:

1. A method of producing 16-alpha-123iodoestradiol-17-beta comprising the steps of:
a) providing as components ¹²³I-sodium iodide, 16-beta-bromoestradiol-17-beta and a phase transfer agent;
b) forming a mixture of the components and heating the mixture in the range of 90 to 120° Centigrade for at least thirty minutes;
c) thereafter separating resulting 16-alpha-123I iodoestradiol-17-beta from the other components of the result of heating the mixture.

2. The method according to claim 1 including the steps of:
a) adding water to the mixture;
b) adding an organic solvent to the mixture; and wherein:
c) the phase transfer agent is benzo-15-crown-ether.

3. The method according to claim 1 wherein the sodium iodide is provided in a mixture with sodium iodate including the step of:
a) prior to forming said mixture, exposing said ¹²³I-sodium iodide to an antioxidant in the presence of heating so as to reduce sodium iodate therein to sodium iodide.

4. The process according to claim 3 wherein:
a) said antioxidant is selected from the group consisting of sodium thiosulphate and sodium metabisulfite.

5. The method according to claim 1 wherein:
a) said mixture is heated in the range from 95 to 110° Centigrade.

6. The method according to claim 1 wherein:
a) said mixture is heated in the range from 100 to 105° Centigrade for a time of approximately 45 minutes.

7. The method according to claim 1 including the step of:
a) utilizing a high pressure liquid chromatograph for said separating.

8. The method according to claim 7 including the step of:
a) inserting the mixture into the chromatograph with a carrier with approximately equal parts by weight water and acetonitrile.

9. The method according to claim 1 including the step of:
a) adjusting the pH of the sodium iodide to within the range of 7.1 to 8 prior to adding the remaining components of the mixture.

10. The method according to claim 9 including the step of:
a) dissolving the mixture in methyl ethyl ketone and water two phase solution prior to insertion into the chromatograph.

11. The method according to claim 1 including the step of:
a) sterilizing the resultant 16-alpha-123I iodoestradiol-17-beta subsequent to separating from the other components of the mixture.

12. A method of making 16-alpha-123iodoestradiol-17-beta with a yield relative to the ¹²³I of greater than 90% in a period of time less than the half life of ¹²³I comprising the steps of:
a) providing a precursor of ¹²³I-sodium iodide and sodium iodate;
b) exposing the precursor to an oxidant selected from the group consisting of sodium thiosulphate and sodium metasulfide with heating in the range of 90 to 120° Centigrade for approximately 15 minutes;
c) thereafter separating the resulting ¹²³I sodium iodide from remaining components of the precursor in a high pressure liquid chromatograph;
d) adjusting the pH of the ¹²³I sodium iodide within the range of from 7.1 to 8 and removing moisture therefrom to dryness;
e) forming a mixture of the precursor with 16-beta-bromoestradiol-17-beta and benzo-15-crown-5-ether and dissolve the mixture in methyl ethyl ketone and water;
f) heating the mixture in the range from 90° C. to 120° C. for a period of time in the range from 30 to 60 minutes;
g) separating the resulting 16-alpha-123iodoestradiol-17-beta from remaining components of the mixture subsequent to heating in the high pressure liquid chromatograph; and
h) sterilizing the separated 16-alpha-123iodoestradiol-17-beta.

13. The method according to claim 12 wherein:
a) the mixture is heated for approximately 45 minutes.

14. A method of producing 16-alpha-haloestradiol-17-beta comprising the steps of:
a) providing a first source of sodium halide;
b) providing a second source of an estradiol with bromine located at the 16-beta position;
c) providing a third source of a phase transfer agent;
d) mixing together said first, second and third sources in the presence of an organic solvent and water to form a mixture;
e) heating the mixture at a temperature in the range of 90 to 120° Centigrade for sufficient time to allow said halide to displace said bromine and join with the estradiol at a 16-alpha position thereof to produce 16-alpha-haloestradiol-17-beta; and
f) separating the resulting 16-alpha-haloestradiol-17-beta from the remaining mixture by use of high pressure liquid chromatography.

15. The method according to claim 14 wherein:
a) the halide is iodine.

16. The method according to claim 14 wherein:
a) the halide is ¹²³I.

17. The method according to claim 16 wherein:
a) the estradiol is 16-beta-bromoestradiol-17-beta.

18. In a method of producing 16-alpha-123I iodoestradiol-17-beta by reaction of 16-beta-bromoestradiol-17-beta with ¹²³I sodium iodide, the improvement comprising:
a) maintaining the reaction temperature between 90 and 120° Centigrade.

19. The method according to claim 18 wherein:
a) the reaction temperature is maintained between 100 and 105° Centigrade for approximately forty-five minutes.
